(19) United States
(12) Patent Application Publication
(10) Pub. No.: US 2017/0322171 A1
(43) Pub. Date: Nov. 9, 2017

(54) GAS-MEASURING CHIP, PORTABLE CHIP MEASUREMENT SYSTEM AND METHOD FOR OPERATING A PORTABLE CHIP MEASUREMENT SYSTEM

(71) Applicant: Dräger Safety AG & Co. KGaA, Lübeck (DE)

(72) Inventors: Wolfgang BÄTHER, Lübeck (DE); Stefan LEHMANN, Uetersen (DE)

(21) Appl. No.: 15/526,073

(22) PCT Filed: Nov. 11, 2015
(86) PCT No.: PCT/EP2015/002257

§ 371 Date: May 11, 2017

(30) Foreign Application Priority Data
Nov. 13, 2014 (DE) 10 2014 016 712.7

(51) Int. Cl. G01N 27/27; G01N 27/414; G01N 27/30; G01N 33/00; G01N 1/22

(52) U.S. Cl. CPC G01N 27/27; G01N 33/0073; G01N 1/2205; G01N 27/30; G01N 27/4141

(57) ABSTRACT

A gas-measuring chip (10), used with a gas-measuring device (100) of a portable chip measurement system, has a carrier (11) and measuring channels (20, 20′, 20″). A regenerable, nonconsumable sensor (30, 30′, 30″) is arranged in each measuring channel. A method includes inserting the gas-measuring chip (10) into the gas-measuring device (100) and connecting one measuring channel of the gas-measuring chip (10) to a pumping system (120, 121) of the gas-measuring device (100). A measurement is carried out with a first measuring channel (20, 20′, 20′) with a switching over to a measuring channel different from the first measuring channel. The sensors (30, 30′, 30″) of the measuring channel used last is regenerated and optionally simultaneously there is a measurement with the measuring channel switched over to. There is a switching over to a measuring channel, which is different from the measuring channel last used for the measurement.

### CROSS REFERENCE TO RELATED APPLICATIONS

This application is a United States National Phase Application of International Application PCT/EP2015/002257 filed Nov. 11, 2015, and claims the benefit of priority under 35 U.S.C. §119 of German Application 10 2014 016 712.7 filed Nov. 13, 2014, the entire contents of which are incorporated herein by reference.

### FIELD OF THE INVENTION

The present invention pertains to a gas-measuring chip, for use with a gas-measuring device of a portable chip measurement system, wherein the gas-measuring chip has a carrier and at least two measuring channels, and the invention relates to a portable chip measurement system as well as to a method for operating a portable chip measurement system.

### BACKGROUND OF THE INVENTION

Prior-art gas-measuring chips (chips) typically have a chip card-like carrier, on which a number of glass capillaries are arranged. Each glass capillary forms here a measuring channel and is typically filled with a detection reagent, through which a gas flow to be analyzed can be sent when the chip is inserted into a corresponding receptacle of a gas-measuring device. The gas measuring chip and the gas-measuring device together form a portable chip measurement system here. If a suitable analyte is contained in the gas flow, which is sent through the capillary, this analyte can react with the detection reagent in the glass capillary, and a color change can occur. This can then be recorded by a corresponding assembly unit of the gas-measuring device, for example, a camera. Such systems are usually used to make it possible to rapidly and reliably determine on the spot whether corresponding limit values of toxic gases or vapors in the ambient air are complied with or exceeded, e.g., at the site of an accident or at workplaces in an industrial environment with potentially high exposure.

The gas-measuring chip and the gas-measuring device are usually configured here such that a measuring channel each, arranged on the chip, i.e., one of the glass capillaries, can be connected during a measurement to a pumping system of the gas-measuring device. The pumping system can then draw or pump the gas sample to be analyzed through the capillary, and the assembly unit intended for the analysis, e.g., the above-mentioned camera, can observe whether a color change takes place in the capillary.

It may, however, be problematic in these systems that only a specific analyte can always be detected by means of a measuring capillary. This particular analyte always depends on the reagents with which the capillary is filled. In addition, each capillary can be used only once. The logistic effort needed for the detection of a plurality of different analytes by means of such a system is correspondingly relatively great. This is especially true if the detection shall be carried out continuously over a longer time period.

Sensor arrays, in which different sensors are arranged on a common carrier and which are simultaneously exposed to a gas sample to be analyzed, are also known for the simultaneous detection and distinction of a plurality of different analytes, in addition to the above-described chip measurement systems (CMS) (K. Albert et al., Chem. Rev., 2000, 100, 2595-2626). However, such arrays may very easily display memory effects, for example, if unexpectedly high analyte concentrations occur. As a consequence, the recovery times (regeneration times) may be very long for the sensor and the waiting times between the measurements may be too long for the user.

### SUMMARY OF THE INVENTION

An object of the present invention is therefore to overcome these and other drawbacks of the state of the art and to provide an improved sensor system, especially an improved gas-measuring chip and an improved chip measurement system. It is desirable, for example, in this connection for the chip measurement system to be able to quantitatively measure a plurality of analytes simultaneously. It is especially desirable for the chip measurement system not to be affected even by unexpectedly high analyte concentrations and for its reliability not to be compromised. The improved sensor system, especially the improved gas-measuring chip and the improved chip measurement system, shall be able to be handled in a simple manner and rapidly as well as manufactured as favorably as possible.

Provisions are made in a gas-measuring chip for use with a gas-measuring device of a portable chip measurement system, wherein the gas-measuring chip has a carrier and at least two measuring channels, for at least one regenerable, nonconsumable sensor to be arranged in each measuring channel.

A gas-measuring chip is defined, in general, as a carrier, preferably a plate-shaped, especially chip card-like carrier, together with the sensors located on the carrier. The sensors may be coordinated with one analyte or a plurality of particular analytes to be detected. A special advantage of the gas-measuring chip according to the present invention is that sensors are arranged in the measuring channels. A sensor may be defined in the broadest sense of the word as a technical component that can qualitatively or quantitatively detect certain physical or chemical properties and/or the material quality of the area surrounding it or can be directly or indirectly converted into an electrical signal that can be subjected to further processing. The analysis of the measured signals can consequently be carried out by transmitting and analyzing the electrical signals sent by the sensors. It is also advantageous in this connection that the sensors of the gas-measuring chips according to the present invention are regenerable and nonconsumable sensors. Nonconsumable sensors require no chemical reagents, which would have to be replenished after a corresponding interaction with an analyte. Also, neither oxygen nor other constituents of the air are needed for a detection reaction or the like. The interaction with an analyte to be detected typically takes place in such sensors due to the adsorption of the analyte on a surface of the sensor, which triggers a corresponding electrical signal. If the sensor is regenerable, it can again return to its initial state after a first interaction with the analyte, so that a corresponding interaction with an analyte can take place repeatedly in a second measurement and a new signal can be triggered. Desorption of the previously adsorbed analyte typically takes place during the return into the initial state. This regenerability may usually cover a nearly unlimited number of consecutive measurements. It is also favorable in this connection if the sensors are continuously measuring sensors.

Another great advantage of a gas-measuring chip according to the present invention is that it can be used as a gas-measuring chip in a system comprising a gas-measuring device and a gas-measuring chip, i.e., it is suitable for use with a gas-measuring device of a portable chip measurement system. A gas-measuring chip is suitable according to the present invention for use with a gas-measuring device of a portable chip measurement system if it can be inserted into a receptacle of a corresponding gas-measuring device, if the measuring channels of the gas-measuring chip can be connected to a pumping system of the gas-measuring device, so that a gas sample to be analyzed can flow through one or more of the measuring channels, and if information or signals obtained from the sensors can be transmitted to the gas-measuring device or can be read from the gas-measuring device. It is thus seen that a gas-measuring device, with which a gas-measuring chip according to the present invention can be used, has a receptacle for the gas-measuring chip, a pumping system and advantageously an analysis unit. It is conceivable that the gas-measuring device also has a second pumping system, optionally a conveying system for the gas-measuring chip in the receptacle as well as additional components, e.g., an energy supply unit, an operator interface and the like. It is advantageous in this connection if a connection device, which is used to connect the measuring channels of the gas-measuring chip to the pumping system, of the gas-measuring device, is formed in the receptacle of the gas-measuring device.

It is thus seen that it is advantageous if the measuring channels are configured to be connected to a pumping system of the gas-measuring device. Each measuring channel has a gas inlet and a gas outlet. A gas sample, which shall be analyzed, can flow into the measuring channel through the gas inlet. The gas sample can again flow out of the measuring channel through the gas outlet. The gas inlet and the gas outlet may be closed with septum seals. These septum seals can be punctured by means of a needle system, which is used as a connection device, when the gas-measuring chip is inserted into the receptacle of the gas-measuring device. The needle system can thus establish a connection to the pumping system of the gas-measuring device. If is therefore advantageous if the gas inlet and the gas outlet of the measuring channels arranged on the gas-measuring chip are arranged on the carrier such that their position corresponds, when the chip is inserted into the gas-measuring device, to the position of the needles in the receptacle of the gas-measuring device.

The measuring channels may be configured, for example, as capillaries. It is also conceivable that the measuring channels are configured as grooves in the surface of the carrier. These grooves may be provided with a cover in such a tight manner that the gas sample can flow through the groove between the gas inlet and the gas outlet in exactly the same way as through a closed tube or capillary. The measuring channels preferably have a linear shape and are likewise preferably arranged parallel to one another on the carrier of the gas-measuring chip. Other arrangements and shapes are, of course, also conceivable. It is always important in this connection that the gas inlet and the gas outlet be able to be connected to the pumping system of the gas-measuring device as described above. The gas inlet and the gas outlet can typically be connected to the pumping system if the pumping system has at least one gas outlet, through which a gas sample can flow out, as well as a gas inlet, through which a gas sample can flow into the measuring channel and then into the pumping system. If the gas inlet and the gas outlet of the measuring channel can be connected to the pumping system, the gas inlet of the measuring channel, the gas inlet of the pumping system, the gas outlet of the measuring channel as well as the gas outlet of the pumping system are each connected to one another fluidically such that the gas sample can correspondingly flow from the pumping system into the measuring channel and back.

It is also advantageous in this connection if the gas-measuring chip has a contact device, which is configured to transmit information of the sensors to an analysis unit of the gas-measuring device. In other words, the gas-measuring chip may have an electronic contact surface for transmitting measured data of the sensors to the gas-measuring device. This contact surface may be, for example, a contact strip or a corresponding printed circuit board, which strip or board is formed in the lateral area of the carrier and is coupled with the sensors, which are arranged in the measuring channels, via electrical connections. Both a common contact surface for all measuring channels and a separate contact surface for each measuring channel may be formed on the gas-measuring chip. If the gas-measuring chip is inserted into the receptacle of a gas-measuring device, the contact device thus configured is preferably in electrically conductive contact with a corresponding opposite contact surface formed in the receptacle of the gas-measuring device. The electrical signals of the sensors can be transmitted in this way from the gas-measuring chip to the gas-measuring device and there further to an analysis unit of the gas-measuring device.

It is, moreover, advantageous in another embodiment variant if the gas-measuring chip has an information carrier, which is suitable for transmitting information via the gas-measuring chip to the gas-measuring device. The information provided by this information carrier may be, for example, information on the age of the chip, the type and quantity of the sensors arranged on the chip, certain measurement conditions and corresponding other information, which the analysis unit needs to be able to analyze the transmitted electrical signals of the sensors. In the simplest case, the information carrier is an optical information carrier, for example, a bar code or a QR code. However, other variants, for example, an RFID tag or data storage devices, are, of course, also conceivable. In any case, it is favorable now if the gas-measuring device has a corresponding reading unit, which can detect the information of the information carrier and transmit it correspondingly to the analysis unit of the gas-measuring device. It is also conceivable that the analysis unit of the gas-measuring device can read the information of the information carrier itself. It is also conceivable in a special embodiment variant that the information carrier is connected to the contact device of the gas-measuring chip. The reading of the information carrier can thus be carried out directly by the analysis unit of the gas-measuring device.

Provisions are made in another preferred embodiment variant of the present invention for a plurality of regenerable, nonconsumable sensors to be arranged in at least one measuring channel. The sensors may be sensitive here each for different analytes. A gas sample, which flows through this measuring channel, can thus be analyzed for a plurality of different analytes simultaneously. It is especially favorable if a plurality of regenerable, nonconsumable sensors are arranged in a plurality of measuring channels or even in each measuring channel. The same sensors or a different selection of sensors may now be arranged in each measuring channel. The number of sensors in the measuring channels of a gas-measuring chip may also correspondingly be equal or different. It is also conceivable that at least two measuring channels each are provided with the same selection and/or number of sensors. The variety of the analytes that are detectable by means of a gas-measuring chip according to the present invention can additionally be increased in this way.

It is also advantageous in each case if the sensors are arranged in a row within a measuring channel. This makes possible, for example, a slim and linear design of the measuring channel and, as a consequence, a space-saving arrangement of a plurality of measuring channels next to one another on the gas-measuring chip.

In addition, it is advantageous if the sensors are selected from among cantilever sensors, surface-acoustic-wave sensors, quartz crystal microbalances, optical systems, field effect transistor systems or the like, preferably field effect transistor systems, especially preferably CCFET sensors, because CCFET sensors offer, in particular, the advantage of being very compact, having a very low intrinsic energy demand, being able to be put into operation within a short time, and being able to be manufactured in large lots according to the MEMS technology. Such CCFET sensors (Capacitively-Controlled Field Effect Transistor Sensors) are typically characterized in that a gas-sensitive layer, on which an analyte can be adsorbed, is coupled capacitively with a field effect transistor via one or more electrodes. The adsorption of the analyte on the gas-sensitive layer then leads to a change in the voltage present on the field effect transistor. This change in voltage can be recognized ultimately as an electrical signal and correspondingly analyzed by the analysis unit of the gas-measuring device.

It is seen that it is favorable if a printed circuit board, on which the sensors of the measuring channel are arranged, is arranged in at least one of the measuring channels. Corresponding electrical signals, which the sensors deliver in case of an interaction with an analyte, can directly or indirectly be transmitted by means of such a printed circuit board to the gas-measuring device. The printed circuit board may be in an electrically conductive connection with the above-described contact device, for example, with a corresponding contact surface.

It is conceivable in one embodiment that at least one measuring channel has a plurality of sensors, which are based on different principles of measurement. It is also conceivable in this connection that all sensors of such a measuring channel are based on different principles of measurement. It is, of course, also conceivable, in addition or as an alternative, that all sensors of one measuring channel are based on the same principle of measurement. A gas-measuring chip according to the present invention may thus have both measuring channels in which all sensors or a plurality of sensors are based on different principles of measurements and at the same time also measuring channels in which all sensors are based on the same principle of measurement.

In another aspect, the present invention makes provisions in a portable chip measurement system with a gas-measuring chip and with a portable gas-measuring device, wherein the gas-measuring device has a receptacle., into which the gas-measuring chip can be inserted, at least one pumping system and an analysis unit, for the gas-measuring chip to be a gas-measuring chip according to the present invention, as described above. It is seen that the great advantage of this portable chip measurement system is again the fact that regenerable, nonconsumable sensors are arranged in the measuring channels of the gas-measuring chip. As is seen from the above explanations, the variety of analytes that can be detected by means of this system can be markedly increased in this way. Another advantage is that the sensors are arranged in a plurality of measuring channels. For example, a memory effect can effectively be avoided in this way. If, for example, a sensor, which is arranged in a first measuring channel, is used for a first measurement or for the start of a measurement and this sensor is suddenly exposed to unexpectedly high analyte concentrations, it is possible in the portable chip measurement system according to the present invention, just as in the gas-measuring chip according to the present invention, to continue the measurement by switching over to the next measuring channel, in which, for example, a similar chip is arranged. This switchover may be carried out, for example, by the chip being displaced within the receptacle of the gas-measuring device, so that another measuring channel with its gas inlet and gas outlet will be connected to the pumping system of the gas-measuring device. It is favorable in this connection if a corresponding gas-measuring device has a conveying device configured for this purpose. It is also possible, as an alternative, that all measuring channels of the gas-measuring chip are connected to the pumping system. However, only the particular gas-measuring channel that is being used for the measurement is supplied now with a corresponding gas sample by the pumping system. All other channels are blinded during this time. In any case, the sensors that are arranged in the respective measuring channels through which no gas sample is flowing can regenerate.

To detect a certain analyte in a gas sample, the portable chip measurement system according to the present invention can thus be used as follows. A suitable gas-measuring chip, namely, a gas-measuring chip according to the present invention, as described above, is first placed or inserted into the receptacle. The information carrier of the gas-measuring chip makes sensor-specific or gas-measuring chip-specific data available for the gas-measuring device. These data may be transmitted to the gas-measuring device, for example, via the contact device of the gas-measuring chip. It is, however, also conceivable that these data are read, as was shown above, by means of a reading device of the gas-measuring device. The data maybe, e.g., the name of the analyte, the range of measurement of the sensor, the duration of the measurement or other data specific of the analyte.

If the gas-measuring chip is inserted into the receptacle, at least a first of the measuring channels of the gas-measuring chip is connected to the pumping system of the gas-measuring device. The gas inlet and the gas outlet of that measuring channel is brought for this into connection with the pumping system such that a gas sample, which the gas-measuring device has drawn in from the surrounding area, can be pumped through the measuring channel. It is favorable in this connection if a needle system, which can puncture, for example, septum seals, which may be arranged over the gas inlet and the gas outlet, as described above, is formed in the area of the receptacle of the gas-measuring device. The needles of the needle system can at the same time establish a flow connection between the pumping system in the gas-measuring device and the measuring channel.

All gas inlets and gas outlets of all measuring channels of the gas-measuring chip are connected to the gas-measuring device via a needle system in a preferred embodiment variant. The gas-measuring device may also have an additional pumping system, which is used before the first measurement for flushing and zeroing the sensors of the gas-measuring chip. This additional pumping system may be provided, for example, with a circulation filter system. It can thus first pump clean, i.e., analyte-free air through the freshly punctured measuring channels. This clean air can then be used to zero the system in a first step. If the system has been cleaned and zeroed, the measurement can be started. It is also conceivable that a calibration of the system is carried out in this manner. The clean air may contain a defined quantity of a certain analyte for this. Such a calibration is advantageously carried out at a temperature that was likewise defined before.

In any case, the actual measurement begins in portable chip measurement systems according to the present invention when the analyte-containing sample air to be analyzed has been drawn through a first measuring channel. Depending on the concentration of the analyte or analytes, the sensor or sensors send(s) the corresponding measured signal. This is transmitted to the gas-measuring device via the contact device of the gas-measuring chip and there to the analysis unit.

In addition to the increased variety of analytes, which was already described above, and the use of memory effects, it is seen that an additional advantage of the portable chip measurement system according to the present invention is that measurements are possible over a rather long, continuous time period even at very high analyte concentrations. If it happens, for example, that a sensor in one of the measuring channels is exposed to a very high analyte concentration, the capacity of the sensor rapidly reaches a certain analyte saturation. Moreover, quantities of analyte contained additionally can then no longer be measured by this sensor and the corresponding electrical signals reach a maximum. After reaching this maximum, this sensor does, however, need a certain recovery time (regeneration time) to be able to interact with more analyte. A direct further use of the sensor is not possible during this time. However, this situation can be recognized from the measured signal curve of the sensor. For example, the analysis unit can thus automatically recognize this situation or a user can recognize this situation by a corresponding display on a display unit or the like. It is possible according to the present invention to switch over to a second measuring channel in such a case. The measurement can then be continued with a sensor arranged in this second measuring channel. The switchover may happen, for example, by the chip being correspondingly displaced in the receptacle. It is also conceivable, as an alternative, that the pumping system of the gas-measuring device supplies the measuring channel with the gas sample via additional needle systems. It is equally conceivable that the switchover takes place automatically, for example, by a corresponding control command, which is outputted by the analysis unit, or manually by an input by the user. No gas sample flows through the first measuring channel any more after this switchover. The contaminated sensor located in this measuring channel can thus regenerate. To support the regeneration of this sensor, the measuring channel may be heated to a suitable temperature TR. If the gas-measuring device has a second pumping system, which contains, for example, calibrating air, as described above, this measuring channel can additionally be flushed with the calibrating air until the contamination disappears.

In another aspect, the present invention thus pertains to a method for operating a portable chip measurement system, comprising the steps of a) inserting the gas-measuring chip into the gas-measuring device and connecting at least one measuring channel of the gas-measuring chip to the pumping system of the gas device, b) carrying out a measurement with a first measuring channel, c) switching over to a measuring channel different from the first measuring channel, d) regenerating the sensors of the measuring channel used previously and optionally carrying out a measurement simultaneously with the measuring channel to which the process was switched over in the preceding step, e) switching over to a measuring channel that is different from the measuring channel used for the measurement in the preceding step, and f) optionally repeating steps d) and e).

To insert the gas-measuring chip into the gas-measuring device corresponding to step a), a gas-measuring chip according to the present invention can simply be pushed into the receptacle of the gas-measuring device. The corresponding measuring channel or the measuring channels is/are connected to the pumping system, as was already described above, for example, by means of a needle system, during the subsequent connection of the at least one measuring channel of the gas-measuring chip to the pumping system of the gas-measuring device.

The measurement corresponding to step b) of the method according to the present invention takes place by the gas sample to be analyzed being drawn or pumped through the desired measuring channel by means of the pumping system of the gas-measuring device. The gas sample now flows past the sensor or sensors arranged in the measuring channel. If corresponding analytes are contained in the gas sample, they can interact with the suitable sensor and a corresponding electrical signal is transmitted to the gas-measuring device via the contact device. The determination and display of the analyte concentration take place during this measurement after a certain time t_{K} as a function of the analyte concentration and the type of the sensor. t_{K} may correspond, for example, to the t90 value, i.e., 90% of the maximum value of a given concentration. t_{K} shall lie within a range of a few seconds to minutes, just as the recovery time (=regeneration time, i.e., the time during which the sensor goes back, for example, to 10% of the maximum). If, however, the measurement system is exposed to a very high analyte concentration, the recovery time may be markedly prolonged, so that no more measurement can be carried out at times over a period of, for example, one hour. The process is switched over in this case corresponding to step c) to a next channel, which can then be used for the measurement.

The switchover corresponding to step c) may take place, for example, by the gas-measuring chip being correspondingly conveyed in the receptacle of the gas-measuring device, so that a new measuring channel is connected to the pumping system of the gas-measuring device. As an alternative, the pumping system may also be switched over within the gas-measuring device, so that the gas sample to be analyzed will flow through a corresponding new measuring channel.

The measurement in step d) and the switchover in step c) of the method according to the present invention take place analogously to the measurement and switchover according to steps b) and c).

After a measuring channel used previously for a measurement has been switched over to a new measuring channel, the sensors arranged in the first-mentioned measuring channel can regenerate corresponding to step d) of the method according to the present invention. In the simplest case, this can happen by the corresponding measuring channel, i.e., the sensors arranged in the measuring channel, no longer being exposed to a corresponding gas sample to be analyzed for a certain time tR. The analyte adsorbed on the surface of the contaminated sensors can again be desorbed during this time until the sensors are again ready for a measurement.

Provisions are made in a preferred embodiment variant of the method according to the present invention for the generation of the sensors in step d) to comprise the heating of the measuring channels. The increase in the temperature in the measuring channel supports the desorption of the analytes adsorbed on the sensor surfaces. This may additionally be supported by flushing with analyte-free air, for example, air that is fed as a calibrating air through a second pumping system.

It is seen that the greater the number of measuring channels available in the gas-measuring chip according to the present invention, the longer may be the duration of regeneration of an individual measuring channel or of the sensors in such a measuring channel corresponding to step d). It is consequently advantageous if the gas-measuring chip has more than two, preferably five, six, seven, eight, nine, ten or more measuring channels. If the measuring time is t_{K}, as was already mentioned above, a channel to be generated can be regenerated over a time period that is obtained from the product of the measuring time t_{K} and the number of channels minus 1. It is seen in this connection that it is also advantageous if the maximum time for heating a measuring channel corresponds to the product t_{K}×M, in which t_{K}=measuring time and M=(number of measuring channels−1).

It additionally proved to be advantageous for the heating if the heating temperature is between 30° C. and 150° C. For example, the temperature TR, which is used for the heating, may be 80° C. It is thus seen that another advantage of the present invention is that the temperature for the heating is about 30° C. to about 150° C., preferably about 40° C. to about 130° C., and especially preferably about 50° C. to about 120° C.

Another great advantage of the gas-measuring chip according to the present invention as well as of the portable chip measurement system according to the present invention and of the method for operating the portable chip measurement system is that in cases in which contamination is very high, the user can replace a first gas-measuring chip used for a measurement with another gas-measuring chip or with a number of additional gas-measuring chips one after another. This can be repeated until the final measurement result is obtained. However, the gas-measuring chips used in the process do not then have to be discarded, but they can regenerate corresponding to step d) of the method according to the present invention. The gas-measuring chips used may be heated for this, for example, overnight.

Further features, details and advantages of the present invention appear from the text of the claims as well as from the following description of exemplary embodiments on the basis of the drawings.

The present invention is described in detail below with reference to the attached figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1a is a schematic view showing an example of a gas-measuring chip according to the present invention;

FIG. 1b is a top view of a measuring channel of a gas-measuring chip according to the present invention, a cross section of which is shown in FIG. 1c;

FIG. 1c is a cross sectional view through the measuring channel shown in FIG. 1b;

FIG. 2a is a schematic view showing an example of a sensor arranged in a measuring channel according to the present invention of a gas-measuring chip, namely a CCFET sensor;

FIG. 2b is a graph showing an example of a typical signal curve of a sensor according to FIG. 2a;

FIG. 3a is a schematic view showing another exemplary embodiment of a gas-measuring chip according to the present invention;

FIG. 3b is a schematic view showing a variant of the exemplary embodiment according to FIG. 3a;

FIG. 3c is a schematic view showing another variant of the exemplary embodiment according to FIG. 3a;

FIG. 4a is a schematic view of a portable chip measurement system according to the present invention with a gas-measuring chip and with a gas-measuring device;

FIG. 4b is a schematic view showing another example of a portable chip measurement system according to the present invention; and

FIG. 5 is a schematic view of the course of the method according to the present invention for operating a portable chip measurement system.

### DESCRIPTION OF THE PREFERRED EMBODIMENTS

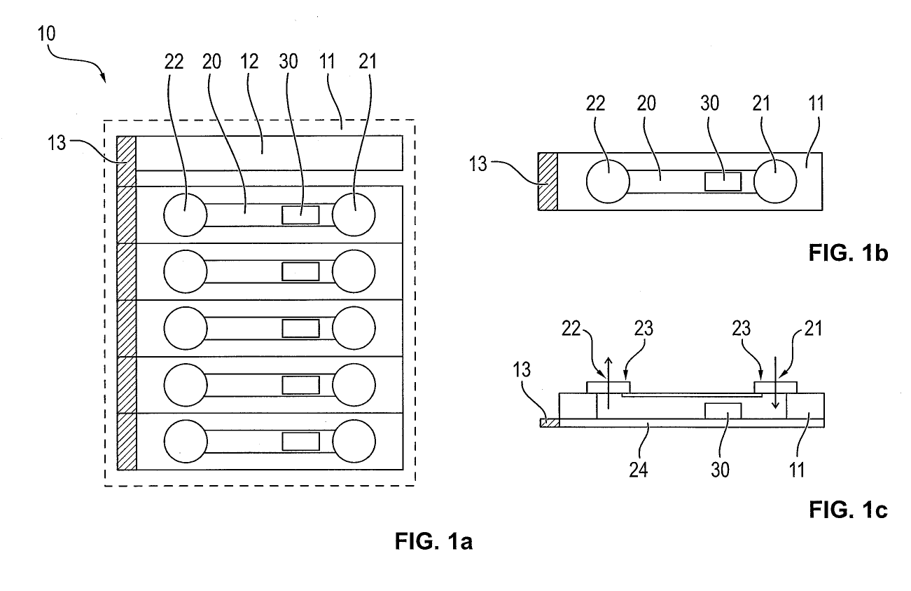

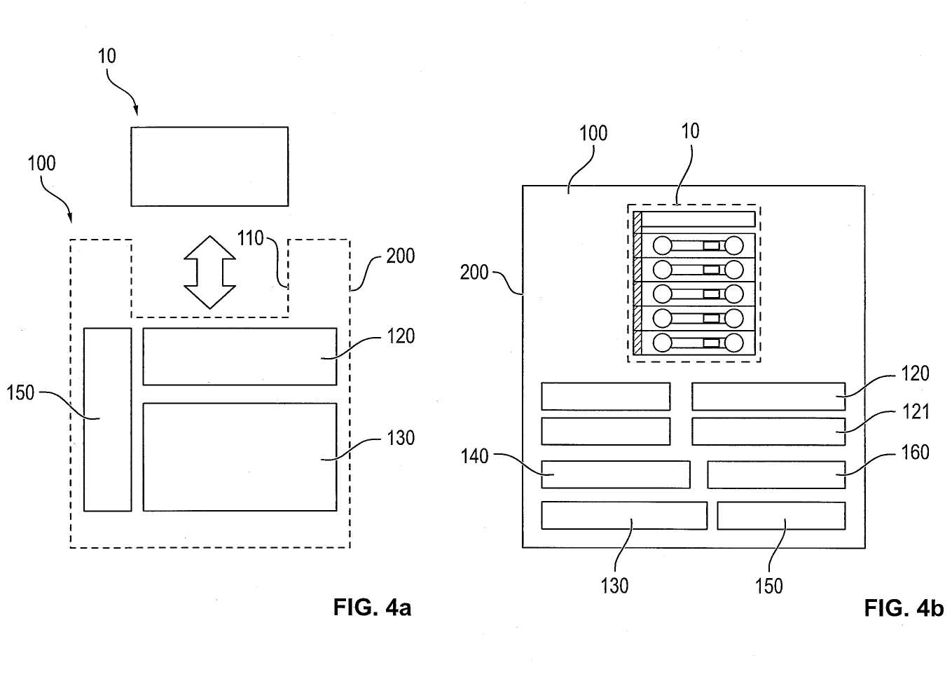

Referring to the drawings, the gas-measuring chip 10 shown in FIG. 1a has a carrier 11, on which a plurality of measuring channels 20 are arranged. At least one sensor 30 is arranged in each measuring channel 20. Each measuring channel 20 has, moreover, a gas inlet 21 and a gas outlet 22. The gas inlet 21 and the gas outlet 22 can be connected to a pumping system 120, 121 of the gas-measuring device 100 when the gas-measuring chip 10 is inserted into a gas-measuring device 100 (cf. FIGS. 4a and 4b).

Furthermore, an information carrier 12 is arranged on the carrier 11 of the gas-measuring chip 10. The information that is contained in or on this information carrier 12 is gas-measuring chip-specific or sensor-specific data, such as the name of the detectable analyte, the measurement range of the sensors of the gas-measuring chip 10, possible or minimal measuring time and the like.

The gas-measuring chip 10 has, furthermore, a contact device 13. This is configured as a lateral strip on the carrier 11. Other embodiment variants, e.g., contact sections, contact pins or the like, are, of course, conceivable.

It is seen in FIG. 1b that each contact device 13 is associated with a measuring channel 20. The contact device 13 is connected to the sensor or sensors 30 arranged in the measuring channel 20 in an electrically conductive manner. This is seen especially in FIG. 1c. The sensor 30 is arranged on a printed circuit board 24. This printed circuit board 24 is, in turn, in contact with the contact device 13. Electrical signals, which are outputted by the sensor 30, can be transmitted to the contact device 13 via the printed circuit board 24.

It is seen, furthermore, in FIG. 1c that the printed circuit board 24 forms a lower limitation of the measuring channel 20 in this exemplary embodiment. The printed circuit board 24 is thus arranged in the measuring channel 20.

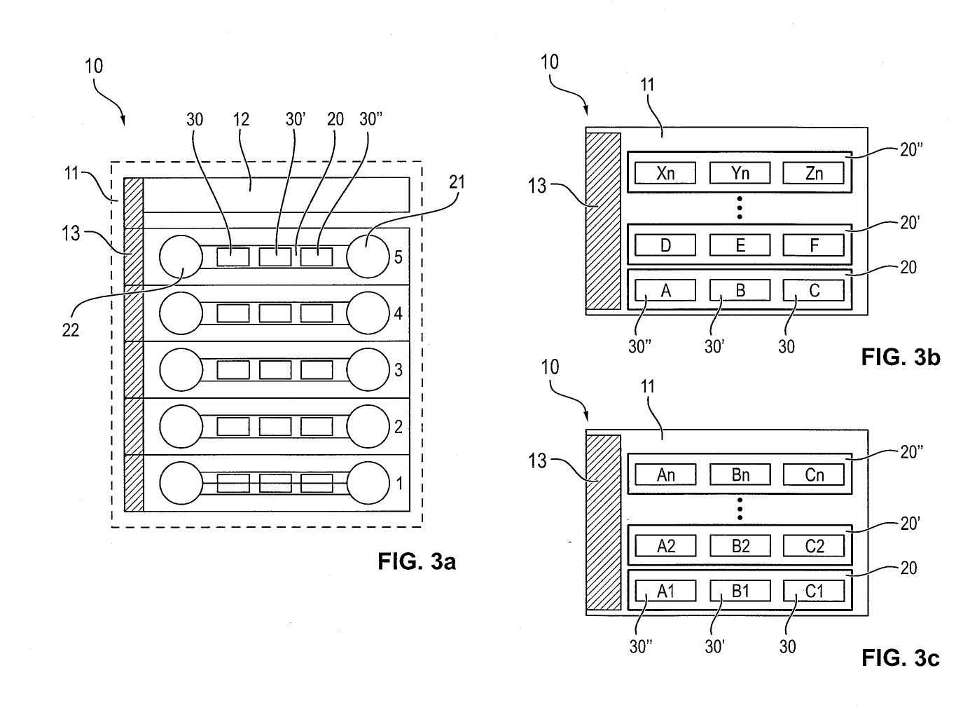

The gas inlet 21 and the gas outlet 22 of the measuring channel 20 are, in addition, closed by septum seals 23. These septum seals 23 can be punctured when the gas-measuring chip 10 is inserted into a gas-measuring device 100. A gas sample will then flow through the gas inlet 21 into the measuring channel 20 and through the measuring channel 20. The gas sample now flows past the sensor 30. A correspondingly suitable analyte, possibly contained in the gas sample, can then interact with the sensor 30. The sensor 30 subsequently sends a correspondingly suitable signal. This signal is transmitted, as was described above, from the printed circuit board 24 to the contact device 13. The gas sample then flows out of the measuring channel through the gas outlet 22. The gas-measuring chip 10, which will be described below and is shown in FIGS. 1a, 1b, and 1c as well as in FIGS. 3a and 3c, is consequently a gas-measuring chip 10 for use with a gas-measuring device 100 of a portable chip measurement system, wherein the gas-measuring chip 10 has a carrier 11 and at least two measuring channels 20 and wherein at least one regenerable, nonconsumable sensor 30 is arranged in each measuring channel 20. The measuring channels 20 of the gas-measuring chip 10 are configured to be connected to a pumping system 120 of the gas-measuring device 100 (cf. FIGS. 4a and 4b). The gas-measuring chip 10 has, furthermore, a contact device 13, which is configured to transmit information of the sensors 30 to an analysis unit 130 (cf. FIGS. 4a and 4b) of the gas-measuring 100. In addition, the gas-measuring chip 10 has an information carrier 12, which is suitable for transmitting information via the gas-measuring chip 10 to the gas-measuring device 100.

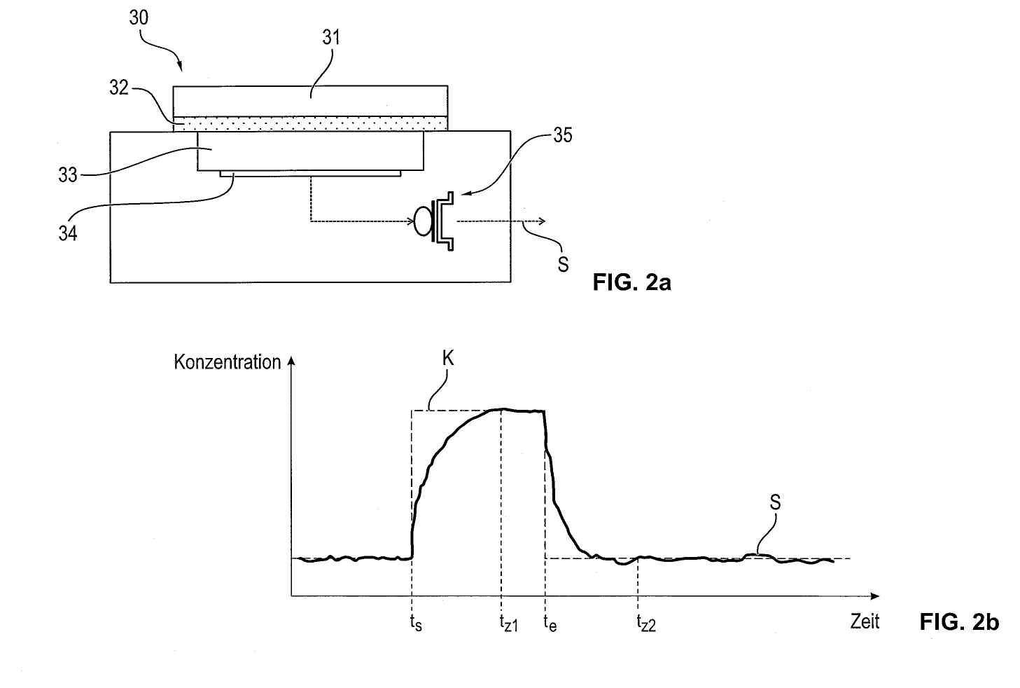

FIG. 2a shows an exemplary embodiment of a sensor 30, which can be used in a gas-measuring chip 10 according to the present invention. FIG. 2a shows a so-called CCFET sensor (Capacitively Controlled Field Effect Transistor sensor). This CCFET sensor has a first electrode 31, which is coated with a gas-sensitive layer 32, and a second electrode 34. An air gap 33 is formed between the first electrode 31 and the second electrode 34. The air gap 33 acts as a dielectric, so that the electrodes 31, 34 act as a capacitor. For an example, an analyte can be carried to the gas-sensitive layer 32 through the air gap 33 and adsorbed there. Such an adsorption leads to a change in the capacity of the capacitor formed by the electrodes 31, 34. This change in capacity can be detected by a field effect transistor 35, which is connected to the capacitor. As a consequence, an electrical measured signal S is outputted. This electrical measured signal S can then be transmitted through the printed circuit board 24, on which the sensor 30 is mounted, to the contact device 13, as was described above.

FIG. 2b shows a typical example of the signal curve of such an electrical measured signal S. The curve K drawn in broken line describes here the concentration curve of the analyte. At the time tS, the electrical measured signal S rises because of the adsorption of the analyte molecules on the gas-sensitive layer 32 in order to reach the maximum at the time tZ1. The analyte concentration is brought to 0 at the time te. The analyte molecules are then desorbed from the surface to be completely desorbed by the time tZ2. The interval between the times te and tZ2 is the period that is called the regeneration time or recovery time of the sensor 30.

FIGS. 3a, 3b and 3c show further exemplary embodiments of a gas-measuring chip 10 according to the present invention. The gas-measuring chip 10 has again a carrier 11 in this case as well, on which a plurality of measuring channels 20, 20′, 20″ are arranged. Each of these measuring channels 20, 20′, 20″ has a gas inlet 21 and a gas outlet 22. In addition, all measuring channels 20, 20′, 20″ are coupled with a contact device 13. This gas-measuring chip 10 has an information carrier 12 as well.

A plurality of sensors 30, 30′, 30″ are arranged in each of the gas-measuring channels 20. These sensors 30, 30′, 30″ may differ in both their principles of measurement and their specificity for a particular analyte to be detected. Different sensors 30, 30′, 30″ are arranged in each measuring channel 20, 20′, 20″ in the exemplary embodiment shown in FIG. 3b. The variety of analytes that can be detected by means of this gas-measuring chip 10 is increased in this way. The information carrier 12 contains information on which type of sensor 30, 30′, 30″ is arranged in which of the measuring channels 20, 20′, 20″. The gas-measuring device 100′, in which such a gas-measuring chip 10 is used, can then specifically select one of the measuring channels 20, 20′, 20″ and send the gas sample to be analyzed through that measuring channel.

Identical sensors 30, 30′, 30″ are arranged in each of the measuring channels 20, 20′, 20″ in the example shown in FIG. 3c. On the one hand, the variety of analytes is increased here, because different sensors 30, 30′, 30″ are arranged in the individual measuring channels 20, 20′, 20″. At the same time, this exemplary embodiment offers the possibility of switching over to another measuring channel 20, 20′, 20″ in case of unexpectedly high analyte concentrations, as was described above. Continuous measurement can be guaranteed in this way even at high analyte concentrations. In addition, this gas-measuring chip is resistant to occurring memory effects.

It is therefore seen that the gas-measuring chip 10 in FIG. 3a or 3b and 3c has at least one measuring channel 20, 20′, 20″, in which a plurality of regenerable, nonconsumable sensors 30, 30′, 30″ are arranged. It seen, furthermore, that the sensors 30, 30′, 30″ are arranged in series within the measuring channels 20, 20′, 20″.

The sensors 30, 30′, 30″ are selected from among cantilever sensors, surface-acoustic wave sensors, quartz crystal microbalances, optical systems, field effect transistor systems or the like. In a special embodiment, the sensors 30, 30′, 30″ are field effect transistor systems, preferably CCFET sensors as described in FIGS. 2a and 2b. The sensors are arranged on a printed circuit board 24 in this gas-measuring chip 10 as well, as was already described in the exemplary embodiment according to FIGS. 1a, 1b and 1c. The printed circuit board 24 is arranged in the respective measuring channel 20, 20′, 20″ in this case as well. All sensors 30 of one measuring channel 20, 20′, 20″ may be based on the same principle of measurement. In an alternative embodiment, each measuring channel 20, 20′, 20″ has a plurality of sensors 30, 30′, 30″, which are based on different principles of measurement.

FIGS. 4a and 4b show each a schematic view of portable chip measurement systems according to the present invention, which comprise each a gas-measuring chip 10 and a gas-measuring device 100. The gas-measuring chip 10 can be replaced depending on the desired analyte, which shall be detected with the corresponding gas-measuring chip 10. The gas-measuring device 100 has a receptacle 110, into which the gas-measuring chip 10 can be inserted. The gas-measuring device 100 has, furthermore, a receptacle 110, into which the gas-measuring chip 10 can be inserted. The gas-measuring device 100 has, furthermore, a pumping system 120 and an analysis unit 130. The pumping system 120 can be connected to the measuring channels 20, 20′, 20″, which are arranged on the gas-measuring chip. In another embodiment, not shown, the gas-measuring device 100 may have a needle system for this, which is arranged in the receptacle 110 and can establish the connection between the gas inlet 21, the gas outlet 22 and the pumping system 120.

The analysis unit 130 of the gas-measuring device 100 according to the present invention can be connected directly or indirectly to the contact device 13 of the gas-measuring chip 10 in any case. The gas-measuring device 100 has a contact element (not shown) for this, which is likewise arranged in the receptacle 110 and which can establish an electrically conductive connection between the contact device 13 and the analysis unit 130. The contact element may be a contact surface, a contact pin or the like.

Furthermore, a reading unit 150 is provided in the embodiment variant of the gas-measuring device 100 shown in FIG. 4a. This reading unit can detect information, which is provided by the information carrier 12 of the gas-measuring chip 10, and correspondingly transmit it to the analysis unit 130. When analyzing the electrical signals received, the analysis unit 130 can then take into account this information, for example, by selecting a corresponding, suitable algorithm in order to display the measurement results or to suitably adapt corresponding measuring times.

The gas-measuring device 100 according to the exemplary embodiment shown in FIG. 4b has, just like the gas-measuring device 100 according to the exemplary embodiment according to FIG. 4a, a receptacle 110 for the gas-measuring chip 10 as well as a first pumping system 120, an analysis unit 130 and a reading unit 150. The gas-measuring device 100 shown in FIG. 4b additionally has a second pumping system 121, a display 160 as well as operating elements 140. The respective components of this gas-measuring device 100 are shown only schematically in FIG. 4b (just like in the case of the gas-measuring device 100 according to FIG. 4a). All components are always arranged in a common housing 200.

The second pumping system 121 shown in the exemplary embodiment according to FIG. 4b is connected to a circulation filter system, not shown. It is used to pump analyte-free air through the measuring channels 20, 20′, 20″ of the gas-measuring chip 10. The gas-measuring chip 10 or the gas-measuring device 100 can be calibrated in this way when inserting the chip 10 or between a plurality of measurements.

The operating elements 140 and the display 160 are used to make possible the comfortable handling of the gas-measuring device 100 or of the portable chip measurement system for a user.

Thus, FIGS. 4a and 4b show a portable chip measurement system with a gas-measuring chip 10 and with a portable gas-measuring device 100, wherein the gas-measuring device 100 has a receptacle 110, into which the gas-measuring chip 10 can be inserted, at least one pumping system 120, 121 and an analysis unit 130, wherein the gas-measuring chip 10 is a gas-measuring chip 10 that is suitable for use with a gas-measuring device of a portable chip measurement system, wherein the gas-measuring chip 10 has a carrier 11 and at least two measuring channels 20, 20′, 20″ and wherein at least one regenerable, nonconsumable sensor 30, 30′, 30″ is arranged in each measuring channel 20, 20′, 20″.

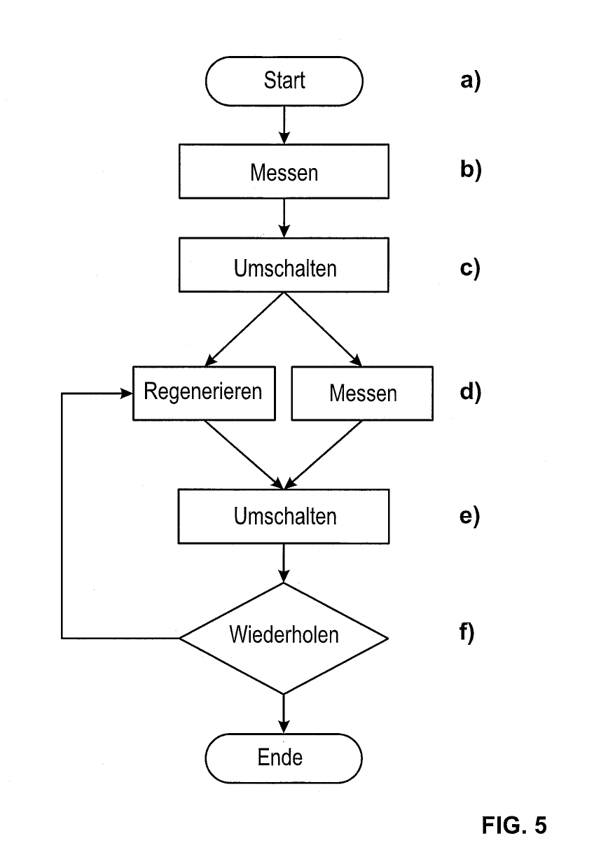

A method as is schematically shown can be carried out with such a system. In a first step a), the gas-measuring chip 10 is inserted into the gas-measuring device 100 for starting the method. At least one of the measuring channels 20, 20′, 20″ of the gas-measuring chip 10 is connected to the pumping system 120, 121 of the gas-measuring device 100 when the gas-measuring chip 10 is inserted. If the gas-measuring device 100 is equipped with a second pumping system 121 corresponding to, for example, FIG. 4b, the gas-measuring chip 10 can first be connected to the second pumping system 121 in step a). This second pumping system 121 then pumps first analyte-free air through the measuring channel or the respective connected measuring channels 20, 20′, 20″ for calibrating or zeroing the gas-measuring chip 10. In a next step, which is not shown in FIG. 5 and is a substep of step a), the first pumping system 120 can then be connected to the respective measuring channels 20, 20′, 20″ in order to proceed with the next step, namely, step b).

It is thus seen that the first step of the method according to the present invention, namely, step a) in a gas-measuring device 100 corresponding to FIG. 4b comprises the insertion of the gas-measuring chip 10 into the gas-measuring device 100 and the connection of at least one measuring channel 20, 20′, 20″ of the gas-measuring chip 10 to the pumping system 120 of the gas-measuring device 100. This step may also comprise the insertion of the gas-measuring chip 10 into the gas-measuring device 100, the connection of a pumping system 121 to the measuring channels 20, 20′, 20″, the calibration of the measuring channels 20, 20′, 20″ and the connection of the pumping system 120 to one or more measuring channels 20, 20′, 20″ after calibration in a gas-measuring device 100 corresponding to FIG. 4b. It is also conceivable in another embodiment variant, not shown, that the first pumping system 120 is used to calibrate the gas-measuring chip 10. Step a) now comprises the corresponding substeps of inserting the gas-measuring chip 10 into the gas-measuring device 100, connection of at least one measuring channel 20, 20′, 20″ to the pumping system 120 and calibration of the gas-measuring system.

Subsequent to step a), a first measurement is carried out with a first measuring channel 20, 20′, 20″ according to step b) of the method shown in FIG. 5. The pumping system 120 pumps for this a gas sample to be analyzed through the respective measuring channel 20, 20′, 20″. The pumping system 120 draws the corresponding gas sample through the gas inlet 21 of the measuring channel 20, 20′, 20″ into the measuring channel 20, 20′, 20″ and removes it through the gas outlet 22. The gas sample to be analyzed now flows past the sensor or sensors 30, 30′, 30″ arranged in the measuring channel 20, 20′, 20″. These sensors can correspondingly interact with analytes that are possibly present and output a signal, e.g., an electrical measured signal S, as is shown in FIG. 2a. This signal is transmitted to the contact device 13 via the printed circuit board 24 and there to the gas-measuring device 100, namely, the analysis unit 130.

If the measuring system is exposed, as was described above, to a very high analyte concentration, or detection of another analyte is desired, for which no suitable sensor 30, 30′, 30″ is arranged in the measuring channel 20, 20′, 20″ used in step b), the process is switched over in the next step c) from the first measuring channel 20, 20′, 20″, which is used in step b), to a new measuring channel 20, 20′, 20″. The sensors 30, 30′, 30″ arranged in the first measuring channel 20, 20′, 20″, which were used for the first measurement in step b), can then regenerate in the next step d), i.e., the analytes adsorbed on their surfaces can now be desorbed. At the same time, a further measurement can be carried out in step d) with the measuring channel 20, 20′, 20″, to which the process was switched over in step c), or the measurement started in step b) with the first measuring channel 20, 20′, 20″ can be continued with this measuring channel 20, 20′, 20″, to which the process was switched over. The switchover in step c) takes place by the chip 10 being conveyed either forward or backward within the receptacle 110 of the gas-measuring device 100. The gas-measuring device 100 may contain a conveying system in an embodiment variant, not shown. As an alternative, the switchover in step c) is brought about by the pumping system 120 being switched over within the gas-measuring device 100 such that the gas sample to be analyzed is drawn through another measuring channel 20, 20′, 20″.

The regeneration of the sensors in step d) comprises, in one embodiment variant, the heating of the measuring channels 20, 20′, 20. The temperature within the respective measuring channel 20, 20′, 20″ is increased for this for a certain time to a temperature of about 30° C. to about 150° C. The temperature of the sensors 30, 30′, 30″, which are arranged in the corresponding measuring channel 20, 20′, 20″, is also increased in the process. In one embodiment variant, the temperature is increased to about 40° C. to about 130° C. In another embodiment variant, the temperature is increased to about 50° C. to about 120° C. In yet another embodiment variant, the temperature is increased to 80° C.

In another embodiment variant, the regeneration of the sensors 30, 30′, 30″ additionally includes the flushing of the measuring channels 20, 20′, 20″ with analyte-free air. Provisions are made in this connection in a first embodiment variant for the regeneration to comprise both the flushing and the above-mentioned heating of the measuring channel 20, 20′, 20″. Provisions are made in another variant for the regeneration to comprise the flushing or heating of the measuring channel 20, 20′, 20″. It is obvious that a plurality of measuring channels 20, 20′, 20″ may also always be regenerated simultaneously in all these variants.

The maximum time for the regeneration and hence for the flushing and/or heating of the measuring channel 20, 20′, 20″ corresponds to the product of the measuring time t_{K} and the number of channels that are arranged on the gas-measuring chip 10 minus 1, i.e., to the product t_{K}×M, in which t_{K}=measuring time and M=(number of measuring channels−1).

If the sensors 30, 30′, 30″ to be regenerated in step d) are fully regenerated and are again ready to be used or the measurement carried out in step d) has ended, the process is again switched over to another measuring channel 20, 20′, 20″ in step e), as is seen in FIG. 5. The switchover is carried out corresponding to the switchover in step c). The process may be switched over now either to the measuring channel 20, 20′, 20″ used in step b) (switched back) or to another measuring channel 20, 20′, 20″, which is likewise arranged on the gas-measuring chip 10.

It is seen, furthermore, in FIG. 5 that according to step f), steps d) and e) can be repeated. The number of repetitions is freely selectable, i.e., steps d) and e) may be carried out one after another as often as desired.

If no repetition according to step f) is desired, the method according to the present invention has ended.

It is seen that the greater the number of measuring channels 20, 20′, 20″ arranged on the respective gas-measuring chip 10, the longer may be the duration of the regeneration of the sensors 30, 30′, 30″. If, for example, a gas-measuring chip 10 has five measuring channels 20, 20′, 20″ and each measuring channel shall be used for a duration of two minutes for the measurement corresponding to step b) or step d), the sensors 30, 30′, 30″ which in the measuring channels 20, 20′, 20″ that are not being used now can be regenerated each for eight minutes without the two-minute measurement frequency having to be reduced.

Therefore, the method shown in FIG. 5 for operating a portable chip measurement system with a gas-measuring chip 10 and with a portable gas-measuring device 100, wherein the gas-measuring device 100 has a receptacle 110, into which the gas-measuring chip 10 can be inserted; at least one pumping system 120, 121 and an analysis unit 130; and wherein the gas-measuring chip 10 is suitable for use with a gas-measuring device 100 of such a portable chip measurement system; wherein the gas-measuring chip 10 has a carrier 11 and at least two measuring channels 20, 20′, 20″, and wherein at least one regenerable, nonconsumable sensor 30, 30′, 30″ is arranged in each measuring channel 20, 20′, 20″, has the following steps: a) inserting the gas-measuring chip 10 into the gas-measuring device 100 and connection of at least one measuring channel 20, 20′, 20″ of the gas-measuring chip 10 to the pumping system 120, 121 of the gas-measuring device 100, b) carrying out a measurement with a first measuring channel 20, 20′, 20″, c) switching over to a measuring channel 20, 20′, 20″ different from the first measuring channel 20, 20′, 20″, d) regeneration of the sensors 30, 30′, 30″ of the measuring channel 20, 20′, 20″ used last and optionally simultaneous performance of a measurement with the measuring channel 20, 20′, 20″ to which the process was switched over in the preceding step, e) switching over to a measuring channel 20, 20′, 20″, which is different from the measuring channel 20, 20′, 20″ used for the measurement in the preceding step, and f) optionally repeating steps d) and e).

It is, furthermore, seen in FIG. 5 that the regeneration of the sensors 30, 30′, 30″ in step d) comprises the heating of the measuring channels 20, 20′, 20″. The maximum time for heating a measuring channel 20, 20′, 20″ corresponds to the product t_{K}×M, in which t_{K}=measuring time and M=(number of measuring channels−1). The temperature for the heating is about 150° C., preferably about 40° C. to about 130° C., and especially preferably about 50° C. to about 120° C.

The present invention is not limited to one of the embodiments described, but may be modified in many different ways. All the features and advantages, including design details, arrangement in space and method steps, which appear from the claims, the description and the drawings, may be essential for the present invention both in themselves and in the many different combinations as well.

While specific embodiments of the invention have been shown and described in detail to illustrate the application of the principles of the invention, it will be understood that the invention may be embodied otherwise without departing from such principles.

1. A gas-measuring chip for use with a gas-measuring device of a portable chip measurement system, the gas-measuring chip comprising:
a carrier;
at least two measuring channels; and
at least one regenerable, nonconsumable sensor arranged in each of the measuring channels.

2. A gas-measuring chip in accordance with claim 1, wherein the measuring channels are configured to be connected to a pumping system of the gas-measuring device.

3. A gas-measuring chip in accordance with claim 1, wherein the gas-measuring chip has a contact device, which is configured to transmit information of the sensors to an analysis unit of the gas-measuring device.

4. A gas-measuring chip in accordance with claim 1, further comprising an information carrier configured for transmitting information on the gas-measuring chip to the gas-measuring device.

5. A gas-measuring chip in accordance with claim 1, wherein the at least one regenerable, nonconsumable sensor comprises a plurality of regenerable, nonconsumable sensors arranged in one of the measuring channels.

6. A gas-measuring chip in accordance with claim 5, wherein a plurality of the regenerable sensors are arranged in series within the one of the measuring channels.

7. A gas-measuring chip in accordance with claim 5, wherein the regenerable sensors are selected from among cantilever sensors, surface-acoustic wave sensors, quartz crystal microbalances, optical systems, and field effect transistor systems.

8. A gas-measuring chip in accordance with claim 1, wherein a printed circuit board, on which the sensors of said measuring channel are arranged, is arranged in at least one of the measuring channels.

9. A gas-measuring chip in accordance with claim 5, wherein at least one of the measuring channels has a plurality of sensors, which are based on different principles of measurement.

10. A gas-measuring chip in accordance with claim 10, wherein all sensors of one measuring channel are based on the same principle of measurement.

11. A portable chip measurement system comprising:
a gas-measuring chip;
a portable gas-measuring device, wherein the gas-measuring device has a receptacle, into which the gas-measuring chip is inserted;
at least one pumping system; and
an analysis unit, wherein the gas-measuring chip comprises:
a carrier;
at least two measuring channels; and
at least one regenerable, nonconsumable sensor arranged in each of the measuring channels.

12. A method for operating a portable chip measurement system comprising a gas-measuring chip, a portable gas-measuring device, wherein the gas-measuring device has a receptacle, into which the gas-measuring chip is insertable, at least one pumping system; and an analysis unit wherein the gas-measuring chip comprises: a carrier; at least two measuring channels; and at least one regenerable, nonconsumable sensor arranged in each of the measuring channels, the method comprising the steps of:
inserting the gas-measuring chip into the gas-measuring device and connecting at least one of the measuring channels of the gas-measuring chip to the pumping system of the gas-measuring device;
carrying out a measurement with a first measuring channel;
switching over to a second measuring channel different from the first measuring channel;
regenerating the sensors of the first measuring channel used last;
carrying out a measurement with the second measuring channel either after the step of regenerating the sensors or simultaneously with the step of regenerating the sensors;
switching over to another measuring channel, which other measuring channel is different from the second measuring channel (20, 20′, 20″) used last.

13. A method in accordance with claim 11, wherein the step of regenerating the sensors comprises heating of the measuring channels.

14. A method in accordance with claim 13, wherein a maximum time for heating a measuring channel corresponds to a product t_{K}×M, in which t_{K}=measuring time and M=a number of measuring channels−1.

15. A method in accordance with claim 13, wherein the temperature for the heating is about 30° C. to about 150° C.

16. A portable chip measurement system in accordance with claim 11, wherein the gas-measuring chip further comprises a contact device configured to transmit information of the sensors to an analysis unit of the gas-measuring device.

17. A portable chip measurement system in accordance with claim 11, further comprising an information carrier configured to transmit information relating to the gas-measuring chip to the gas-measuring device.

18. A portable chip measurement system in accordance with claim 11, wherein the at least one regenerable, nonconsumable sensor comprises a plurality of regenerable, nonconsumable sensors, each being arranged in one of the measuring channels.

19. A portable chip measurement system in accordance with claim 18, wherein the plurality regenerable sensors are arranged in series within the one of the measuring channels.

20. A portable chip measurement system in accordance with claim 11, wherein the regenerable sensors are selected from among cantilever sensors, surface-acoustic wave sensors, quartz crystal microbalances, optical systems, and field effect transistor systems.
